(19) United States
(12) Patent Application Publication
(10) Pub. No.: US 2022/0361429 A1
(43) Pub. Date: Nov. 17, 2022

(54) TORRADOVIRUS RESISTANCE GENE

(71) Applicant: RIJK ZWAAN ZAADTEELT EN ZAADHANDEL B.V., De Lier (NL)

(72) Inventors: Agathe Anna NOWOSIELSKI, De Lier (NL); Dörthe Bettina DRÄGER, De Lier (NL); Roy KOOPMANS, De Lier (NL)

(21) Appl. No.: 17/747,113

(22) Filed: May 18, 2022

Related U.S. Application Data
(63) Continuation-in-part of application No. PCT/EP2020/087448, filed on Dec. 21, 2020.
Application No. PCT/EP2020/087448 is a continuation-in-part of application No. PCT/EP2019/086815, filed on Dec. 20, 2019.

(51) Int. Cl. A01H 1/00; A01H 6/82; A01H 1/04

(52) U.S. Cl. CPC A01H 1/126; A01H 6/82; A01H 1/045

(57) ABSTRACT

The present invention relates to a modified FBXL13 gene which leads to a plant showing resistance to a virus belonging to the Torradovirus genus. The invention also relates to a plant belonging to the Solanaceae family comprising the modified FBXL13 gene. The modified FBXL13 gene of the invention provides a plant showing resistance to a virus belonging to the Torradovirus genus when compared to plants not comprising the modified FBXL13 gene. The invention further relates to the use of the modified FBXL13 gene for the identification and development of a plant belonging to the Solanaceae family showing resistance to a virus belonging to the Torradovirus genus.

### RELATED APPLICATIONS AND INCORPORATION BY REFERENCE

This application is a continuation-in-part application of international patent application Serial No. PCT/EP2020/087448 filed 21 Dec. 2020, which published as PCT Publication No. WO 2021/123429 on 24 Jun. 2021, which claims benefit of international patent application Serial No. PCT/EP2019/086815 filed 20 Dec. 2019.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing which has been submitted electronically and is hereby incorporated by reference in its entirety. Said ASCII copy was created Dec. 21, 2020, is named Y7954-00520SL.txt and is 108,710 bytes in size.

### FIELD OF THE INVENTION

The present invention relates to a gene that is able to confer resistance to a virus belonging to the Torradovirus genus. The invention also relates to a plant belonging to the Solanaceae family which may comprise a gene that is able to confer resistance to a virus belonging to the Torradovirus genus, and to methods for identifying and developing such a plant. Furthermore, the invention relates to the use of plants, seeds and propagation material derived from such plants as a germplasm in a breeding program.

### BACKGROUND OF THE INVENTION

Commercial vegetable production is affected by many conditions that influence the growth and development of the crop. The choice of the grower for a certain variety is a determining factor, and genetics of the selected variety forms the basis for the result that can be achieved. In addition, there are many external factors that influence the outcome give, for example, the disease pressure. Growing conditions like climate, soil, and the use of inputs like fertilizer play a major role. There are various ways of cultivating other crops, among which, the most common are: open field, greenhouse and shade house production. The presence of pests and diseases also affects the total yield that can be reached.

Breeding for multiple disease and pest resistances is an important aspect in providing varieties for multiple growing systems and climates. These diseases can be the result of attacks of either nematodes, bacteria, fungi, insects and/or viruses.

More specifically, resistance to viruses in an important and desirable trait in commercial tomato production. Around 2006, a new virus was discovered in tomato, especially on plants that were grown in Spain. As the virus caused symptoms that could not be attributed to known viruses, the new virus was tentatively named Tomato torrado virus (ToTV) and was also subject of a patent application (WO2006/085749)

Citation or identification of any document in this application is not an admission that such document is available as prior art to the present invention.

### SUMMARY OF THE INVENTION

It is one object of the present invention to provide a plant belonging to the Solanaceae family showing resistance to a virus belonging to the Torradovirus genus. This object has been achieved by providing a modified FBXL13 gene that when present in the genome of a plant belonging to the Solanaceae family confers resistance to a virus belonging to the Torradovirus genus. A plant belonging to the Solanaceae family that has the mutated gene is in particular resistant to a virus belonging to the Torradovirus genus.

The characterisation of the modified FBXL13 gene in the present research was performed in tomato (Solanum lycopersicum). This enabled the identification of further crops belonging to the Solanaceae family having a FBXL13 gene, which when modified leads to a plant which confers resistance to a virus belonging to the Torradovirus genus when compared to a plant not having the modified FBXL13 gene. These crops include those belonging to the family of Solanaceae, such as pepper (Capsicum annuum) eggplant (Solanum melongena) and potato (Solanum tuberosum).

The invention thus relates to a modified FBXL13 gene which may comprise a modification in the wild type FBXL13 nucleotide sequence which leads to a modification in the wild type FBXL13 amino acid sequence.

The modified FBXL13 gene may be an exogenous FBXL13 gene introduced into a plant by a transgenic method or a cisgenic method. The modified FBXL13 gene of the invention may also be used for developing a plant that confers resistance to a virus belonging to the Torradovirus genus, which may comprise the introduction of a modified exogenous FBXL13 gene by a transgenic or a cisgenic method.

The modified FBXL13 gene may be part of a gene construct, which gene construct may comprise a selectable marker, a promoter sequence, a FBXL13 gene sequence, and a terminator sequence.

The present invention is widely applicable to all plant species that have a functional orthologue of the FBXL13 gene in their genome, i.e. an orthologue that performs the same or a similar biological function. Identification of FBXL13 orthologues, i.e. FBXL13 genes in other species, can be performed in many crops, methods of which are known in the art. The present invention can for instance be applied to a plant belonging to a species selected from the group consisting of Solanum lycopersicum, Solanum tuberosum, Solanum melongena and Capsicum annuum.

Accordingly, the present invention relates to a modified FBXL13 gene which may comprise a modification in the wild type FBXL13 nucleotide sequence of SEQ ID NOS: 1-4 which leads to a modification in the wild type FBXL13 amino acid sequence of SEQ ID NOS: 5-8 respectively. When such a modified FBXL13 gene is present in the genome of a plant belonging to the Solanaceae family, it confers resistance to a virus belonging to the Torradovirus genus.

FIGS. 1-4 show the wild type FBXL13 nucleotide sequences SEQ ID NOS:1-4 of Solanum lycopersicum, Solanum melongena, Solanum tuberosum and Capsicum annuum, respectively. FIGS. 5-8 show the wild type FBXL13 amino acid sequences SEQ ID NOS: 5-8 of Solanum lycopersicum, Solanum melongena, Solanum tuberosum and Capsicum annuum, respectively.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U. S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 Solanum lycopersicum FBXL13 wildtype coding sequence, SEQ ID NO: 1. The wildtype nucleotide is “T”, indicated between brackets and bold here at position 732.

FIG. 2 Solanum melongena FBXL13 wildtype coding sequence, SEQ ID NO: 2.

FIG. 3 Solanum tuberosum FBXL13 wildtype coding sequence, SEQ ID NO: 3.

FIG. 4 Capsicum annuum FBXL13 wildtype coding sequence, SEQ ID NO: 4.

FIG. 5 Solanum lycopersicum FBXL13 wildtype amino acid sequence, SEQ ID NO: 5. The wildtype amino acid is “Y”, indicated between brackets and bold here at 244 amino acids from the start.

FIG. 6 Solanum melongena FBXL13 wildtype amino acid sequence, SEQ ID NO: 6.

FIG. 7 Solanum tuberosum FBXL13 wildtype amino acid sequence, SEQ ID NO: 7.

FIG. 8 Capsicum annuum FBXL13 wildtype amino acid sequence, SEQ ID NO: 8.

FIG. 9 Solanum lycopersicum FBXL13 modified coding sequence, SEQ ID NO: 9. The nucleotide between brackets and bold indicates the position of the deletion, 732 nucleotides from the start. Due to the deletion, the modified coding sequence now has a nucleotide “A”, as shown here. This modification results in a gene with a premature STOP codon.

FIG. 10 Solanum lycopersicum FBXL13 modified coding sequence, SEQ ID NO: 10. The nucleotide between brackets and bold indicates the position of the SNP, 731 nucleotides from the start. The SNP is “C”, as shown here. In this modified coding sequence, the wildtype nucleotide “T” at position 732 is deleted. Both the SNP and the deletion result in a gene with a premature STOP codon.

FIG. 11 Solanum lycopersicum FBXL13 modified coding sequence, SEQ ID NO: 11. The nucleotide between brackets and bold indicates the position of the SNP, 731 nucleotides from the start. The SNP is “C”, as shown here. In this modified coding sequence, the wildtype nucleotide “T” at position 732 is present. The presence of only the SNP will not result in a gene with a premature STOP codon.

FIG. 12 Solanum lycopersicum FBXL13 modified amino acid sequence, SEQ ID NO: 12. This sequence represents a truncated protein when compared to the wildtype amino acid sequence with SEQ ID NO: 5 of FIG. 5.

FIG. 13 Solanum lycopersicum FBXL13 modified amino acid sequence, SEQ ID NO: 13. This sequence represents a truncated protein when compared to the wildtype amino acid sequence with SEQ ID NO: 5 of FIG. 5.

FIG. 14 Solanum lycopersicum FBXL13 modified amino acid sequence, SEQ ID NO: 14. This sequence does not represent a truncated protein but as a result of the SNP, the wildtype amino acid “Y” at position 244 is changed into “5”.

### DETAILED DESCRIPTION OF THE INVENTION

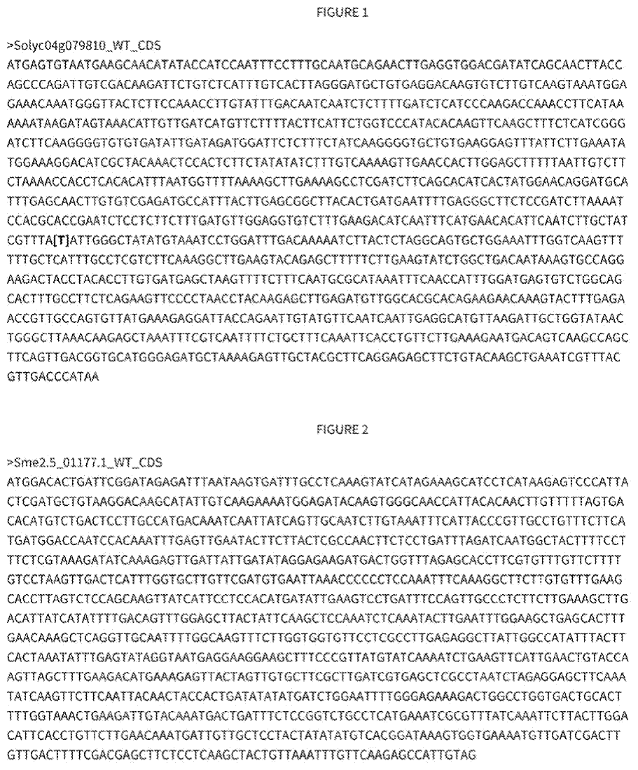

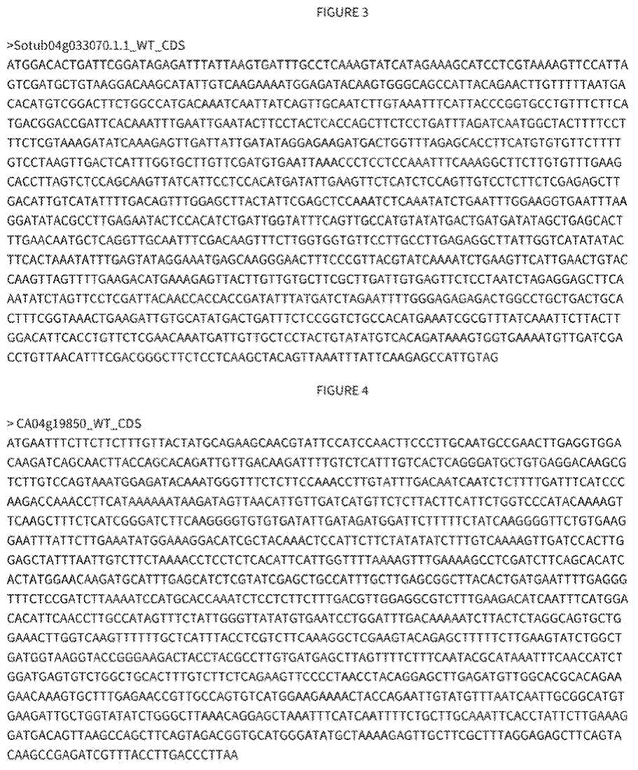

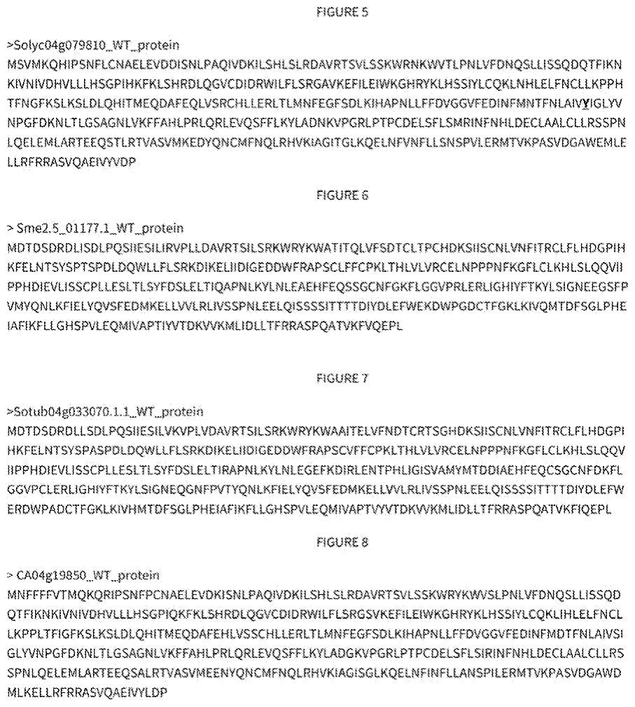

As used herein, ‘wild type’ refers specifically to the naturally occurring, non-modified form of the FBXL13 gene, the naturally occurring, non-modified form of the nucleotide sequence of FBXL13 and the naturally occurring, non-modified form of the FBXL13 amino acid sequence. The naturally occurring, non-modified forms of the FBXL13 gene and FBXL13 protein of several crops are shown in FIGS. 1-4 and FIGS. 5-8 respectively.

The modification leading to the modified FBXL13 gene may be selected from a modification that changes the mRNA level of the FBXL13 gene, a modification that changes the FBXL13 protein structure and/or levels, and/or a modification that changes the FBXL13 protein activity.

One aspect of the invention relates to a modified FBXL13 gene, which may comprise a mutation as compared to its wild type genomic sequence, which mutation leads to a change in the FBXL13 protein and/or protein activity, wherein the modified FBXL13 gene is capable of conferring the phenotype of resistance to a virus belonging to the Torradovirus genus.

In an embodiment, the modified FBXL13 gene results in a decreased activity of the corresponding protein. In this context this “decreased activity” shall mean a decrease in activity of a FBXL13 protein when compared to a corresponding wild type plant cell or a corresponding wild type plant. Decrease shall in one aspect comprise an entire knock-out of gene expression, or the production of a loss of function or of a decreased function of the FBXL13 protein, e.g. a truncated FBXL13 protein may have lost function or show a decreased function. A decrease in activity can be a decrease in the expression of a gene encoding a FBXL13 protein (also referred to as knock-down), or a knock-out of the expression of a gene encoding a FBXL13 protein and/or a decrease in the quantity of a FBXL13 protein in the cells or a decrease of function or loss of function in the enzymatic activity of a FBXL13 proteins in the cells.

In one embodiment, the mutation in the nucleotide sequence is a Single Nucleotide Polymorphism (SNP).

In one embodiment, the mutation in the nucleotide sequence is an indel.

In one embodiment of the present invention, the change in the nucleotide sequence leads to a substitution in the amino acid sequence.

In another embodiment of the present invention, the mutation in the nucleotide sequence leads to a premature stop codon in the amino acid sequence. An amino acid sequence which may comprise a premature stop codon (when compared to its wild type sequence) is also referred to as a truncated amino acid sequence.

In the research leading to the invention, several tomato plant lines and varieties (from wild as well as domesticated species) were found to be resistant to infection with the Tomato Torrado virus. A comparison of susceptible lines and varieties and the resistant ones showed that in tomato a deletion of one thymine (T) at position 732 in the coding sequence of Solyc04g79810 (ITAG2.4 annotation) is affecting an essential codon: whereas a nucleotide deletion in the reference sequence leads to a (premature) STOP codon (TAA), the additional nucleotide (thymine) present in susceptible lines ensures that the coding sequence is extended when compared with the reference sequence. The inventors concluded that a tomato plant which has in its genome the deletion at position 732 of Solyc04g79810 (ITAG2.4 annotation) as present in resistant lines and varieties is resistant to infection with the Tomato Torrado virus.

In several resistant tomato plants, also an additional SNP at position 731 of Solyc04g79810 was found. In this position the nucleotide changed from A to C. Despite the fact that this SNP circumvents the initial STOP codon introduced by the deletion at position 732 by changing the codon from TAA to TCA, another premature STOP codon residing 5 amino acids downstream at position 249 still provides a truncated protein that confers resistance to infection of a tomato plant with Tomato Torrado virus. However, a tomato plant which may comprise the SNP at position 731 but not comprising the deletion at position 732 is likely not resistant to infection with the Tomato Torrado virus since in the absence of the deletion no premature STOP codon is generated.

The invention also relates to all modifications upstream of position 732 of the coding sequence that result in a truncated protein. The protein of the invention (truncated at position 244) already is able to trigger resistance of the invention in a plant belonging to the Solanaceae family. Also, tomato plants comprising the slightly extended, yet truncated protein at position 249 were found to be resistant. Therefore, all truncated versions which are truncated on or before position 249 will have the same effect and thus result in resistance to a virus belonging to the Torradovirus genus. The positions mentioned above are referring to tomato (Solanum lycopersicum). For a crop other than Solanum lycopersicum, positions corresponding to these positions apply.

In a specific embodiment, the modified tomato FBXL13 gene includes a tomato FBXL13 gene which may comprise a deletion on position 732 of SEQ ID NO: 1. or on a position corresponding thereto in the FBXL13 gene of other crops, wherein the indel constitutes a single base pair (T) deletion in the nucleotide sequence on that position. This modification leads to a truncated version of the FBXL13 protein on position 244 of the wildtype amino acid tomato sequence of SEQ ID NO: 5, or on a position corresponding thereto, in other crops. In tomato, this deletion results in an amino acid change from Y to STOP. In other crops the change in nucleotide and amino acid may be different. The modified Solanum lycopersicum FBXL13 coding sequence is indicated with SEQ ID NO: 9 in FIG. 9. The modified Solanum lycopersicum FBXL13 amino acid sequence, resulting from the deletion and comprising the premature stop codon is indicated with SEQ ID NO: 12 and indicated in FIG. 12.

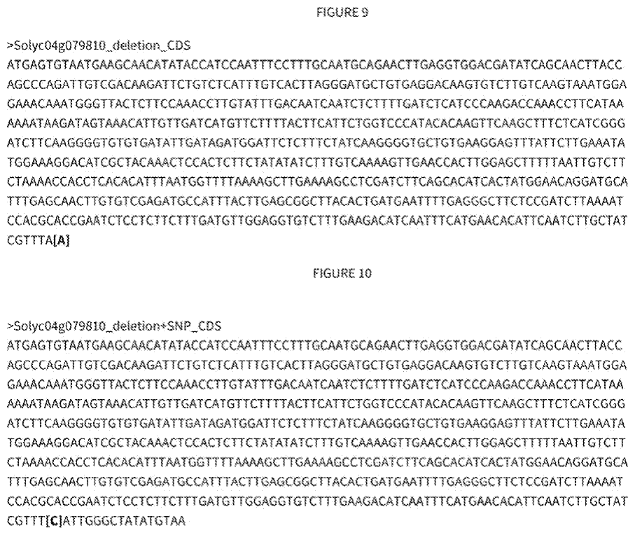

In another embodiment, the modified Solanum lycopersicum FBXL13 nucleotide sequence of SEQ ID NO: 9 further includes a SNP on position 731 of SEQ ID NO: 9, or, for a crop other than Solanum lycopersicum, on a position corresponding to position 731 of the Solanum lycopersicum nucleotide sequence of: 9. This modification leads to disruption of the STOP codon on position 244 of the Solanum lycopersicum amino acid sequence of SEQ ID NO: 12, or, for a crop other than Solanum lycopersicum, on a position corresponding to position 244 of the Solanum lycopersicum amino acid sequence of SEQ ID NO: 12. The further modified Solanum lycopersicum FBXL13 coding sequence is indicated with SEQ ID NO: 10 in FIG. 10. The modified Solanum lycopersicum FBXL13 amino acid sequence, resulting from the SNP and also which may comprise a premature STOP codon at position 249 is indicated with SEQ ID NO: 13 and shown in FIG. 13. Preferably, in Solanum lycopersicum the SNP is a nucleotide change is from A to C and results in the amino acid change is from STOP to S. In other crops the change in nucleotide and amino acid may be different.

Modifications to the gene of the invention may be recessive, dominant or intermediate. In case of a recessive trait, the modification of the gene needs to be present in homozygous state for the trait to be completely visible. The modifications described herein are recessive and thus only confer resistance to a virus belonging to the Torradovirus genus if both alleles of the gene have the modification. Modifications that are dominant or intermediate can also be visible in heterozygous state. The heterozygous phenotype of an intermediate trait lies between the phenotypes of the homozygous dominant and the homozygous recessive genotypes. These types of modifications are also part of the invention.

In a further embodiment of the present invention, the modification in the amino acid sequence is a substitution as indicated on position 244 of the tomato amino acid sequence of SEQ ID NO: 13 or the introduction of a stop codon as indicated at position 244 of the tomato amino acid sequence of SEQ ID NO:12. In case of a crop other than tomato, the modifications in the amino acid sequence are located on a position corresponding to position 244 of the tomato amino acid sequence of SEQ ID NO: 5.

The amino acid substitution and the introduction of a stop codon caused by the mutation and the deletion, respectively of the current invention was found to be present on position 244 of the tomato amino acid sequence of SEQ ID NO: 5, or, in case of a crop other than tomato, on a position corresponding to position 244 of the wild type amino acid sequence SEQ ID NO: 5 of tomato. This nucleotide mutation and the nucleotide deletion are considered to be non-conservative, and the subsequent amino acid change can be considered non-conservative.

Amino acid changes in a protein occur when the mutation of one or more base pairs in the coding DNA sequence result in an altered codon triplet that encodes a different amino acid. Not all point mutations in the coding DNA sequence lead to amino acid changes, due to the redundancy of the genetic code. Mutations in the coding sequence that do not lead to amino acid changes are called “silent mutations”. Other mutations are called “conservative”, they lead to the replacement of one amino acid by another amino acid with comparable properties, such that the mutations are unlikely to change the folding of the mature protein, or influence its function. As used herein a “non-conservative amino acid change” refers to an amino acid that is replaced by another amino acid that has different chemical properties that may lead to decreased stability, changed functionality and/or structural effects of the encoded protein.

In a further preferred embodiment of the present invention, the modification in the amino acid sequence is an introduction of a STOP codon at position 244 of the wildtype amino acid tomato sequence of SEQ ID NO: 5, resulting in a truncated protein. This modified amino acid tomato sequence is referred to as SEQ ID NO: 12. Additionally, the amino acid tomato sequence may comprise an alternative modification resulting in a substitution, consisting of a change from to Y (Tyrosine) to S (Serine) at position 244 of the wildtype amino acid tomato sequence of SEQ ID NO: 5. However, the alternative modification in combination with the deletion also leads to a truncated protein as due to the SNP a STOP codon is now present at position 249 as indicated in the amino acid tomato sequence of SEQ ID NO: 13. When compared to the modified amino acid sequence of SEQ ID NO: 5, the modification on position 731 disrupts the STOP codon at position 244 that resulted from the deletion into S (Serine).

The invention further relates to a plant belonging to the Solanaceae family which may comprise a modified FBXL13 gene as defined herein that confers resistance to a virus belonging to the Torradovirus genus.

A plant belonging to the Solanaceae family which may comprise the modified FBXL13 gene shows a resistant phenotype, i.e. shows resistance to a virus belonging to the Torradovirus genus, compared to an isogenic plant of the same species not comprising the modified FBXL13 gene. For example, a Solanum melongena plant, a Solanum lycopersicum plant, a Solanum tuberosum plant and a Capsicum annuum plant which may comprise the modified FBXL13 gene shows resistance to a virus belonging to the Torradovirus genus. These plants are therefore particularly suitable for cultivation under conditions, where an increased risk to infection with a virus belonging to the Torradovirus genus.

The plant of the invention which may comprise the modified FBXL13 gene either homozygously or heterozygously may be a plant of an inbred line, a hybrid, a doubled haploid, or a plant of a segregating population.

A plant may have the modified FBXL13 gene in heterozygous state. Such a plant may be a potential source of the gene and when crossed with another plant that optionally also has the modified gene either homozygously or heterozygously can result in progeny plants that have the modified gene homozygously or heterozygously and show the trait of Torradovirus resistance. Preferably, a plant may comprise the modified FBXL13 gene in homozygous state.

In one embodiment, the invention relates to a mutant plant belonging to the Solanaceae family which may comprise a modified FBXL13 gene, wherein the modified FBXL13 gene confers resistance to a virus belonging to the Torradovirus genus to the plant as compared to a plant comprising a non-modified FBXL13 gene.

In another embodiment, the invention relates to a mutant plant belonging to the Solanaceae family which may comprise a modified FBXL13 gene, wherein the modified FBXL13 gene confers resistance to a virus belonging to the Torradovirus genus to the plant, as compared to a plant comprising a non-modified FBXL13 gene, due to a targeted, induced mutation. Genome editing methods such as the use of a CRISPR system could be employed to obtain a plant of the invention.

In another embodiment, the invention relates to a mutant plant belonging to the Solanaceae family which may comprise a modified FBXL13 gene, wherein the modified FBXL13 gene confers resistance to a virus belonging to the Torradovirus genus to the plant, as compared to a plant which may comprise a non-modified FBXL13 gene, due to an induced mutation. Conventional mutagenesis methods, making use of chemical compounds or physical means could be used to obtain such a plant of the invention.

In another embodiment, the invention relates to a mutant plant belonging to the Solanaceae family which may comprise a modified FBXL13 gene, wherein the modified FBXL13 gene confers resistance to a virus belonging to the Torradovirus genus to the plant, as compared to a plant which may comprise a non-modified FBXL13 gene, due to a mutation induced by technical means.

The invention also relates to a method for the production of a plant belonging to the Solanaceae family having the modified FBXL13 gene that leads to a Torradovirus resistant phenotype by using a seed that may comprise the modified FBXL13 gene for growing the said plant.

The invention further relates to a method for the production of a plant belonging to the Solanaceae family which may comprise the modified FBXL13 gene by using tissue culture of plant material that carries the modified FBXL13 gene in its genome.

The invention furthermore relates to a method for the production of a plant belonging to the Solanaceae family which may comprise the modified FBXL13 gene which leads to a Torradovirus resistant phenotype, by using vegetative reproduction of plant material that carries the modified FBXL13 gene in its genome.

The invention further provides a method for the production of a plant belonging to the Solanaceae family which may comprise the modified FBXL13 gene by using a doubled haploid generation technique to generate a doubled haploid line from a plant belonging to the Solanaceae family which may comprise the modified FBXL13 gene.

The invention further relates to a seed of a plant belonging to the Solanaceae family which may comprise the modified FBXL13 gene of the invention, wherein the plant that can be grown from the seed shows a Torradovirus resistant phenotype.

The invention also relates to a method for seed production which may comprise growing plants belonging to the Solanaceae family from seeds of the invention, allowing the plants to produce seeds by allowing pollination to occur, and harvesting those seeds. Production of the seeds is suitably done by crossing or selfing. Seeds produced in that manner result in a Torradovirus resistant phenotype of the plants grown thereof.

The invention furthermore relates to a hybrid seed and to a method for producing such a hybrid seed which may comprise crossing a first parent plant belonging to the Solanaceae family with a second parent plant belonging to the Solanaceae family and harvesting the resultant hybrid seed, wherein said first parent plant and/or said second parent plant has the modified FBXL13 gene of the invention. Preferably, a hybrid seed may comprise the modified FBXL13 gene of the invention in homozygous state. A hybrid plant belonging to the Solanaceae family resulting from growing the resulting seed that may comprise the modified FBXL13 gene of the invention, showing the Torradovirus resistant phenotype of the invention is also a plant of the invention. Preferably, such a hybrid plant may comprise the modified FBXL13 gene of the invention in a homozygous state.

Another aspect of the invention relates to propagation material capable of developing into and/or being derived from a plant belonging to the Solanaceae family which may comprise a modified FBXL13 gene, wherein the plant shows a Torradovirus resistant phenotype, compared to an isogenic plant of the same species not comprising the modified FBXL13 gene, wherein the propagation material may comprise the modified FBXL13 gene of the invention and wherein the propagation material is selected from the group consisting of a microspore, pollen, an ovary, an ovule, an embryo, an embryo sac, an egg cell, a cutting, a root, a hypocotyl, a cotyledon, a stem, a leaf, a flower, an anther, a seed, a meristematic cell, a protoplast and a cell, or tissue culture thereof.

The invention thus further relates to parts of a plant belonging to the Solanaceae family that are suitable for sexual reproduction. Such parts are for example selected from the group consisting of a microspore, pollen, an ovary, an ovule, an embryo sac, and an egg cell. In addition, the invention relates to parts of a plant belonging to the Solanaceae family that are suitable for vegetative reproduction, which is in particular a cutting, a root, a stem, a cell or a protoplast. The parts of the plants as mentioned above are considered propagation material. The plant that is produced from the propagation material may comprise the modified FBXL13 gene that confers the Torradovirus resistant phenotype.

According to a further aspect thereof, the invention provides a tissue culture of a plant belonging to the Solanaceae family which may comprise the modified FBXL13 gene of the invention, which is also propagation material. The tissue culture may comprise regenerable cells. Such tissue culture can be selected or derived from any part of the plant, in particular from a leaf, pollen, an embryo, a cotyledon, a hypocotyl, a meristematic cell, a root, a root tip, an anther, a flower, a seed and a stem. The tissue culture can be regenerated into a plant carrying the modified FBXL13 gene of the invention, which regenerated plant expresses the trait of the invention and is also part of the invention.

The invention furthermore relates to a cell of a plant belonging to the Solanaceae family as claimed. Such cell may be either in isolated form or may be part of the complete plant or parts thereof and then still constitutes a cell of the invention because such a cell harbours the modified FBXL13 gene that leads to the Torradovirus resistant phenotype. Each cell of a plant of the invention carries the modified FBXL13 gene that leads to the Torradovirus resistant phenotype. Such a cell of the invention may also be a regenerable cell that can be used to regenerate a new plant of the invention.

The invention also relates to a cell of a plant belonging to the Solanaceae family, which plant may comprise the modified FBXL13 gene and the Torradovirus resistant phenotype. The invention also relates to a cell of a plant that may comprise the modified FBXL13 gene and the Torradovirus resistant phenotype, which plant is obtainable by crossing a plant belonging to the Solanaceae family which may comprise the modified FBXL13 gene and selecting for a plant that shows the Torradovirus resistant phenotype.

The invention also relates to the harvested part of a plant belonging to the Solanaceae family which may comprise the modified FBXL13 gene. Such a harvested part is for example the fruit or the tuber of such a plant.

The invention further relates to a harvested part of a plant belonging to the Solanaceae family which may comprise the modified FBXL13 gene, wherein the part is a food product, optionally a processed food product made thereof, which may comprise the modified FBXL13 gene.

According to another aspect, the invention relates to a part of a fruit which is produced by a plant of the invention. Parts of a fruit of the invention are optionally in processed form, for example the part is a slice, a part of a slice, a cube, or any other part of a fruit. Processed fruit parts of the invention can optionally be mixed with other vegetables, fruits, or other food products. Processed food products are optionally packaged in a container or bag, and such packaged food product which may comprise a fruit or part of a fruit of the invention is also a part of the invention. The food product or harvested part may have undergone one or more processing steps. Such a processing step might comprise, but is not limited to any one of the following treatments or combinations thereof: cutting, washing, cooking, steaming, baking, frying, pasteurizing, freezing, grinding, extracting oil, pickling, or fermenting. The processed form that is obtained is also part of this invention. The invention also relates to the use of a fruit produced by a plant of the invention for further processing into a processed food product by cutting, slicing, peeling, or any other treatment, optionally followed by mixing with one or more other food products. A preferred food product which may comprise fruits—or parts thereof—of the plant of the invention is a salad, wherein the fruits may optionally be mixed with leaves of for example lettuce, spinach, endive, chicory, beet, Swiss chard, etc, and/or with other fruits and/or vegetables.

The invention further relates to the use of the modified FBXL13 gene for the development of a plant belonging to the Solanaceae family showing a Torradovirus resistant phenotype. A skilled person is familiar with introducing a new trait into a plant already having other desired agricultural properties, for instance by introgression. Introgression can be done by means of standard breeding techniques, wherein selection can be done either phenotypically or with the use of markers or a combination thereof.

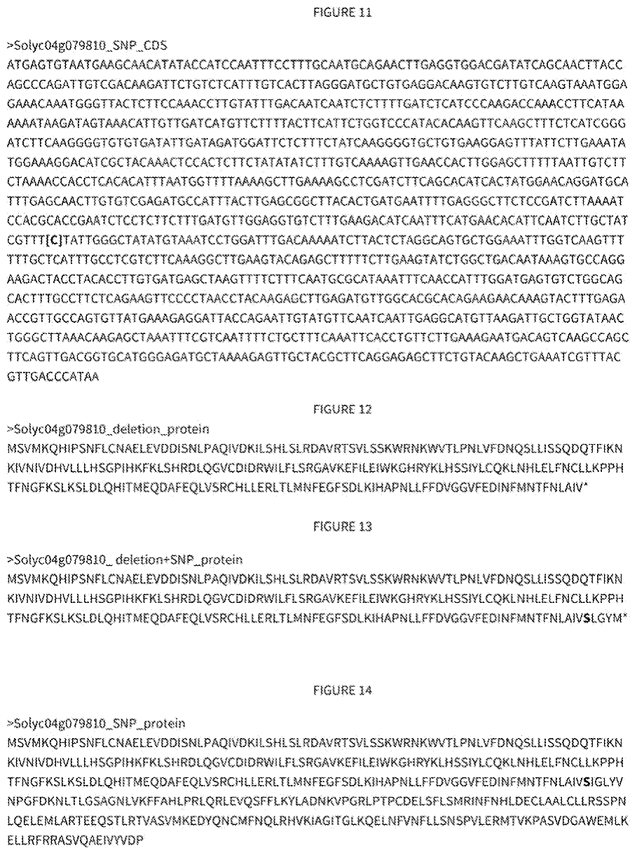

The invention also relates to a marker for the identification of a modified FBXL13 gene in a plant, which marker may comprise any of the modifications in a FBXL13 gene as described herein and can thereby identify and/or detect said modifications. A marker of the invention is in particular a marker which may comprise, and is thereby suitable for identifying and/or detecting, a SNP modification, i.e. a polymorphism, as presented in FIGS. 9-11. The use of such marker for identification and/or detection of a modified CCA gene is also part of this invention.

The invention further relates to the use of the modified FBXL13 gene, or a part thereof which may comprise the modification, as a marker for identifying a plant showing showing a Torradovirus resistant phenotype.

The ‘use for identifying’ or a ‘method for identifying’ as used in the current application may comprise the use of the described indel and/or SNP in the FBXL13 gene as a marker. The invention also relates to other markers that can be developed based on a modification, including the indel or the SNP, in the FBXL13 gene, as well as to other markers that can be developed based on the wildtype sequence of the FBXL13 gene.

In general, to identify a plant belonging to the Solanaceae family showing the Torradovirus resistance phenotype, it is thus determined in the FBXL13 gene whether there is a deletion of a T on position 732 of SEQ ID NO: 1 of tomato, or in case of crop other than tomato, on a position corresponding thereto.

In addition to determining this deletion, it could also be determined whether a mutation from A to C on position 731 of SEQ ID NO: 1 of tomato, or in case of crop other than tomato, on a position corresponding thereto, is present.

The invention relates to a method for producing a plant belonging to the Solanaceae family which shows resistance to a virus belonging to the Torradovirus genus, which may comprise reducing the endogenous level of FBXL13 protein in the plant by modifying the endogenous FBXL13 gene to become a non-functional FBXL13 gene.

The invention also relates to a method for producing a plant belonging to the Solanaceae family which shows resistance to a virus belonging to the Torradovirus genus, which may comprise reducing the endogenous level of FBXL13 protein in the plant by reducing the expression of the FBXL13 gene of the plant by gene silencing or RNAi.

The invention further relates to a method for producing a plant belonging to the Solanaceae family which shows resistance to a virus belonging to the Torradovirus genus, which may comprise reducing the endogenous level of FBXL13 protein in the plant by mutagenic treatment of the seed, in particular by chemical or physical means.

The invention also relates to a method for producing a plant belonging to the Solanaceae family which shows resistance to a virus belonging to the Torradovirus genus, which may comprise reducing the endogenous level of FBXL13 protein in the plant by means of CRISPR.

Mutation(s) in the gene and/or regulatory sequences thereof are preferably introduced randomly by means of one or more chemical compounds, such as ethyl methane sulphonate (EMS), nitrosomethylurea, hydroxylamine, proflavine, N-methyl-N-nitrosoguanidine, N-ethyl-N-nitrosourea, N-methyl-N-nitro-nitrosoguanidine, diethyl sulphate, ethylene imine, sodium azide, formaline, urethane, phenol and ethylene oxide, and/or by physical means, such as UV-irradiation, fast neutron (FN) exposure, X-rays, gamma irradiation, and/or by insertion of genetic elements, such as transposons, T-DNA, retroviral elements. Mutagenesis also may comprise the more specific, targeted introduction of at least one modification by means of homologous recombination, oligonucleotide-based mutation introduction, zinc-finger nucleases (ZFN), transcription activator-like effector nucleases (TALENs) or Clustered Regularly Interspaced Short Palindromic Repeat (CRISPR) systems.

The invention further relates to a method for producing a plant belonging to the Solanaceae family which shows a Torradovirus resistant phenotype which may comprise;

a) crossing a plant belonging to the Solanaceae family which may comprise the modified FBXL13 gene of the current invention with a plant belonging to the Solanaceae family not comprising the modified FBXL13 gene, to obtain an F1 population;

b) performing one or optionally more rounds of selfing and/or crossing a plant from the F1 to obtain a further generation population; and

c) selecting a plant that has a Torradovirus resistant phenotype and the modified FBXL13 gene of the invention.

The invention also relates to a method for producing a plant belonging to the Solanaceae family which shows a Torradovirus resistant phenotype which may comprise;

a) crossing a plant belonging to the Solanaceae family which may comprise the modified FBXL13 gene of the current invention with another plant belonging to the Solanaceae family which may comprise the modified FBXL13 gene, to obtain an F1 population;

b) optionally performing one or more rounds of selfing and/or crossing a plant from the F1 to obtain a further generation population; and

c) selecting a plant that has a Torradovirus resistant phenotype and the modified FBXL13 gene of the invention.

The invention additionally provides a method of introducing another desired trait into a plant belonging to the Solanaceae family which shows a Torradovirus resistant phenotype which may comprise:

a) crossing a plant belonging to the Solanaceae family which may comprise the modified FBXL13 gene of the current invention, with a second plant belonging to the Solanaceae family that exhibits a desired trait to produce F1 progeny;

b) selecting an F1 progeny that shows a Torradovirus resistant phenotype and/or comprise the modified FBXL13 gene of the current invention and the desired trait;

c) crossing the selected F1 progeny with either parent plant, to produce backcross progeny;

d) selecting backcross progeny that shows a Torradovirus resistant phenotype and/or may comprise the modified FBXL13 gene; and

e) optionally repeating steps c) and d) one or more times in succession to produce selected fourth or higher backcross progeny that shows a Torradovirus resistant phenotype. The invention includes a plant produced by this method.

The word “trait” in the context of this application refers to the phenotype of the plant. In particular, the word “trait” refers to a Torradovirus resistant phenotype as a result of the presence of the modified FBXL13 gene.

The invention further relates to a marker for identifying a plant belonging to the Solanaceae family showing resistance to a virus belonging to the Torradovirus genus, which may comprise the modified FBXL13 gene, or a part thereof that may comprise the modification. Preferably, the modification is a nucleotide deletion on or before position 732 of SEQ ID NO:1 of tomato, or, in case of a crop other than tomato, on or around or before a position corresponding to position 732 of SEQ ID NO:1 of tomato, which modification leads to the introduction of a premature stop codon in the corresponding protein, and thus in a truncated protein.

The invention also relates to a method for selecting a plant belonging to the Solanaceae family showing a Torradovirus resistant phenotype from a population of plants, which may comprise detecting the absence of an thymine (T) on position 732 of the tomato nucleotide sequence of SEQ ID NO:1, or, for a crop other than tomato, on a position corresponding to position 732 of the tomato nucleotide sequence of SEQ ID NO: 1, in the genome of a plant of a population of plants, and selecting a plant lacking a thymine (T) on position 732 of SEQ ID NO:1, as shown in SEQ ID NO: 1, or, for a crop other than tomato, on a position corresponding to position 732 of the tomato nucleotide sequence of SEQ ID NO: 1.

The terms “mutant”, “mutation”, “modification”, “modified”, “mutated FBXL13 gene” and “modified FBXL13 gene” as used herein refer to nucleotide changes and amino acid changes to the wild type FBXL13 gene thereof that lead to a modified version of the wild type gene. The modification can be any modification, including but not limited to a single nucleotide polymorphism (SNP), missense mutation, nonsense mutation, insertion or deletion.

More particularly, nucleotide changes and amino acid changes to the wild type FBXL13 gene or protein that lead to a modified version of the wild type FBXL13 gene or protein can lead to a non-functional FBXL13 gene or protein, i.e. a FBXL13 gene encoding a truncated, incomplete FBXL13 protein.

As used herein a “non-functional gene” is a gene that is not expressed or leads to the expression of a non-functional protein. The non-functionality can be the result of a modification to the gene or the complete absence of the gene by deletion. A non-functional gene can be a gene that is deleted and therefore absent. When a gene is deleted, the whole sequence corresponding to the gene is absent in the genome of the plant and leads therefore to non-functionality of the gene. Alternatively, part of the gene that is important for the gene expression such as the promoter, can also be deleted and lead to a non-functional gene. A non-functional gene can also be a gene that leads to a truncated version of the encoded protein that is no longer biologically active or not fully biologically active.

The term “wild type” as used herein refers in general to the form of an organism, gene, protein, or trait as it would occur in nature, as opposed to a mutated or modified form. In the context of the present invention, the term “wild type” refers to an organism, gene or protein that is susceptible and/or provides susceptibility to a virus belonging to the Torradovirus genus.

The term “Single Nucleotide Polymorphism” (“SNP”) is a variation in a single nucleotide that occurs at a specific position in the genome, or more specifically, in the gene of a plant.

The term “missense mutation” is a change in one nucleotide that results in the substitution of one amino acid for another in the protein encoded by a gene.

The term “nonsense mutation” is a change in one nucleotide that results in a premature stop codon. This type of mutation results in a truncated protein that may be non-functional when compared to the wild type protein, depending on the position of the truncation.

The term “premature stop codon” is used to indicate the presence of a stop codon at a position upstream of the stop codon as it is present in the wild type amino acid sequence. Such a premature stop codon results in a truncated, incomplete and usually non-functional protein when compared to the wild type protein.

The term “coding sequence” as used herein is the portion of the nucleotide sequence of a gene composed of exons that code for the protein.

The term “indel” is a term from the molecular biology field and is used to refer to an insertion or deletion of one or more base(s) in the genome. It is classified among small genetic variations, measuring from 1 to 10.000 base pair(s) in length.

The modified FBXL13 gene is also referred to herein as “the gene of the invention”, “the modified FBXL13 gene”, or “the modified FBXL13 gene of the invention”. These terms are used interchangeably herein. As used herein the phrase “the modified FBXL13 gene” is intended to encompass the FBXL13 gene with any modification that leads to resistance to a virus belonging to the Torradovirus genus.

The terms “resistant phenotype” or “resistance” are used interchangeably herein and refer to a phenotype of a plant resistant to infection with virus belonging to the Torradovirus genus. It can be determined whether a plant shows the resistance of the invention according to the method described in patent application WO2006/085749, which discloses a method for identifying a Tomato torrado virus (ToTV) resistant tomato (Solanum lycopersicum) plant. A plant is resistant according to the invention when, after exposing a plant or plant part to ToTV, disease symptoms in said plant of plant part remain absent or are delayed in expression in severity relative to a susceptible control plant that is grown under the same conditions and exposed to ToTV in the same disease test. According to the UPOV Guidelines for the conduct of tests for distinctness, uniformity and stability (TG/44/11 Rev. 2) the tomato variety Daniela is susceptible. Daniela is thus used in the disease test as the susceptible control.

Prior to the bioassay, the virus is multiplicated in Nicotiana tabacum cv. Xanthi that is inoculated in the cotyledon/first leaf stage. After three weeks, the inoculum is harvested when the inoculated plants for multiplication show yellow leafs and show systemic infection. Tomato seeds (including the abovementioned susceptible control) are sown and grown in a glasshouse at a temperature of 23° C. during the day and 21° C. during the night, while the light period is set at 16 hours/day. Tomato plants are inoculated 14 days after sowing, when the cotyledons are fully grown. Optionally, plants can be re-inoculated 21 days after sowing on first true leaves. First observations are performed 7 days after (re-)inoculation, a second observation can be performed 14 days after (re-)inoculation, while final observations should be performed 18 days after (re-) inoculation. Observations comprise visual inspection of the plants taking into account the disease symptoms provided below. The evaluation of resistance should be compared with the results of the susceptible control.

Disease symptoms of systemic infection include necrotic spots at the top of the plant, starting at the base of the leaves of a leaflet. Necrotic spots expand and are surrounded by a light-green or yellow area. These necrotic lesions finalize in burn-like full necrosis of plant material, and eventually in death of the plant. Not all systemic infected leaves show symptoms, however, the virus can be detected in these leaves, for instance by electron microscopy. Fruits infected with ToTV exhibit necrotic rings.

The term “a virus belonging to the Torradovirus genus” may include, but is not limited to the following viruses: Carrot torrado virus 1, Lettuce necrotic leaf curl virus, Motherwort yellow mottle virus, Squash chlorotic leaf spot virus, Tomato marchitez virus, Tomato apex necrosis virus, Tomato torrado virus, Tomato torrado virus ESP/PRIToTV0301, Cassava Torrado-like virus, Red clover torrado virus 1, Tomato chocolate spot virus, Tomato chocolate virus, Tomato necrotic dwarf virus.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1

### Creation of a Plant with a FBXL13 Gene Mutation; Genetic Modification of Plants by Ethyl Methane Sulfonate (ems) and Identification of Plants which have the Mutated FBXL13 Gene

Seeds of the relevant crop, in particular tomato, are treated with EMS by submergence of approximately 5000 seeds into an aerated solution of 0.5% (w/v) EMS during 24 hours at room temperature. The treated seeds are germinated on paper in a small plastic container and the resulting plants are grown and self-pollinated in a greenhouse to produce seeds. After maturation, these seeds are harvested and bulked in one pool. The resulting pool of seeds is used as starting material to identify the individual plants that show resistance to infection with Tomato Torrado virus.

The FBXL13 mutants which are obtained are grown in a greenhouse in order to produce lines by self-fertilisation. Tomato plant lines are analysed to confirm the resistance to infection with Tomato Torrado virus. When a line is segregating for infection with Tomato Torrado plants are selected and after an additional cycle of inbreeding FBXL13 lines are selected. FBXL13 mutants are identified by their resistance to Tomato Torrado virus in comparison with susceptible control lines.

### Example 2

### Identification of the FBXL13 Gene Modification in Solanum lycopersicum

Several crossing populations of tomato (Solanum lycopersicum) plants were tested for resistance against Tomato Torrado virus. Through the use of molecular markers, a QTL was identified, located on chromosome 4. The region of interest was narrowed down to 34 kbp. This region comprised 9 genes.

Sequenced parental lines were subjected to Tomato Torrado virus disease tests, of which a great number was found being resistant with the exception of 2 parental lines.

Whole genome sequencing data of the susceptible parent line TO 5029 and resistant lines were compared to the region of interest (SL2.50ch04:64096226..64132851) using the IGV-tool. Sequences of these lines were extracted and used for further analysis using CloneManager software.

Several SNPs and indels were found. To check for potentially effective mutations (in the coding regions of the genes), mRNA from all the genes in the region was extracted from the Solgenomics website (www.solgenomics.net) and used for alignment with the susceptible and resistant lines.

In the parent line TO 5029, a total of 20 mutations was identified, whereas in the other resistant parent line (S 15R.1640001) a total of 16 mutations was found. For all SNPs and indels, molecular markers were designed in order to investigate which mutation correlates best to the Torrado virus resistance. Of these markers, the molecular marker SL09491 correlated almost 100%.

A SNP was found on position 731 of locus Solyc04g79810. After analysis, it was concluded that this SNP has the following effect: whereas the nucleotide present in susceptible lines provides a Tyrosine (Y) amino acid at that position in the subsequently extended protein, the SNP provides a Serine (S) on that position.

In the research leading to the invention, several tomato plant lines and varieties (from wild as well as domesticated species) were found to be resistant to the Tomato Torrado virus. A further comparison of the susceptible lines and varieties and the resistant ones showed that a deletion within Solyc04g79810 (ITAG2.4 annotation) is affecting an essential codon: whereas a nucleotide deletion in the reference sequence leads to a (premature) STOP codon, the additional nucleotide (thymine) present in susceptible lines ensures that the coding sequence is extended when compared with the reference sequence.

The inventors came to the following conclusion: a tomato plant resistant to infection with the Tomato Torrado virus always has a deletion at position 732 of Solyc04g79810 (ITAG2.4 annotation) as present in resistant lines and varieties. In other resistant plants, also an additional SNP can be found. Despite the fact that this SNP circumvents the initial STOP codon (introduced by the deletion), another premature STOP codon residing 5 amino acids upstream still provides a truncated protein that confers resistance to infection with the Tomato Torrado virus to a tomato plant.

### Example 3

### Identification of Crops Comprising the FBXL13 Gene

A Basic Local Alignment Search Tool (BLAST) program was used to compare the FBXL13 nucleotide sequence of SEQ ID NO: 1 and the amino acid sequence of SEQ ID NO: 5 against the nucleotide coding sequences and protein sequences of other crop plants. This resulted in the identification of candidate FBXL13 orthologous genes in other plants. The multiple sequence alignment of the FBXL13 coding sequence confirmed that these were orthologous FBXL13 genes. Multiple sequence alignment of the amino acid sequences confirmed that these were orthologous FBXL13 proteins.

The invention is further described by the following numbered paragraphs:

1. A modified FBXL13 gene comprising a modification in the wild type FBXL13 nucleotide sequence of SEQ ID NOS: 1-4 which leads to a change in the wild type FBXL13 amino acid sequence of SEQ ID NOS: 5-8, wherein said modified FBXL13 gene when present in the genome of a plant belonging to the Solanaceae family confers resistance to a virus belonging to the Torrado virus genus to said plant.

2. The modified FBXL13 gene of paragraph 1, wherein the modification of the nucleotide sequence is a SNP or indel that leads to a change in the amino acid sequence of the FBXL13 protein.

3. The modified FBXL13 gene of paragraph 1 or 2, wherein the modification of the nucleotide sequence of SEQ ID NOS: 1-4 leads to a premature STOP codon.

4. The modified FBXL13 gene of paragraph 1 or 2, wherein the modification of the nucleotide sequence of SEQ ID NOS: 1-4 leads to a substitution in the amino acid sequence of SEQ ID NOS: 5-8.

5. The modified FBXL13 gene of any of the paragraphs 1-3, wherein the modification of the nucleotide sequence is a deletion of the nucleotide on position 732 of the Solanum lycopersicum nucleotide sequence of SEQ ID NO: 1, or, for a crop other than Solanum lycopersicum, on a position corresponding to position 732 of the Solanum lycopersicum nucleotide sequence of SEQ ID NO: 1, leading to a truncated version of the Solanum lycopersicum amino acid sequence of SEQ ID NO: 5, or, for a crop other than Solanum lycopersicum, on a position corresponding to position 244 of the Solanum lycopersicum amino acid sequence of SEQ ID NO: 5.

6. The modified Solanum lycopersicum FBXL13 gene of paragraph 5, wherein the nucleotide sequence is depicted by SEQ ID NO: 9, which leads to the amino acid sequence of SEQ ID NO: 12.

7. The modified Solanum lycopersicum FBXL13 gene of paragraph 5, wherein the nucleotide sequence further may comprise a SNP on position 731 of the Solanum lycopersicum nucleotide sequence of SEQ ID NO: 1, or, for a crop other than Solanum lycopersicum, on a position corresponding to position 731 of the Solanum lycopersicum nucleotide sequence of SEQ ID NO: 1, leading to an amino acid substitution on position 244 of the Solanum lycopersicum amino acid sequence of SEQ ID NO: 5, or, for a crop other than Solanum lycopersicum, on a position corresponding to position 244 of the Solanum lycopersicum amino acid sequence of SEQ ID NO: 5.

8. The modified Solanum lycopersicum FBXL13 gene of paragraph 7, wherein the nucleotide sequence is depicted by SEQ ID NO: 10, which leads to the amino acid sequence of SEQ ID NO: 13.

9. Plant belonging to the Solanaceae family, comprising the modified FBXL13 gene of any one of the paragraphs 1-8.

10. Plant of paragraph 9, wherein the modified FBXL13 gene results in the plant showing resistance to a virus belonging to the Torradovirus genus as compared to an isogenic plant of the same species not comprising the modification of the FBXL13 gene.

11. Plant of paragraph 9 or 10, wherein the plant belongs to a species selected from the group consisting of Solanum melongena, Solanum lycopersicum, Solanum tuberosum and Capsicum annuum.

12. A seed of a plant of any of the paragraphs 9 to 11, comprising a modified FBXL13 gene.

13. A seed capable of developing into a plant of any of the paragraphs 9 to 11, comprising a modified FBXL13 gene.

14. Propagation material capable of developing into and/or being derived from a plant of any of the paragraphs 9 to 11, wherein the propagation material comprises the modified FBXL13 gene of any of the paragraphs 1 to 8 and wherein the propagation material is selected from a microspore, pollen, ovary, ovule, embryo, embryo sac, egg cell, cutting, root, hypocotyl, cotyledon, stem, leaf, flower, anther, seed, meristematic cell, protoplast, or cell, or tissue culture thereof.

15. Marker for identifying a plant belonging to the Solanaceae family showing resistance to a virus belonging to the Torradovirus genus, comprising the modified FBXL13 gene of any one of the paragraphs 1-8 or a part thereof that comprises the modification.

16. Marker of paragraph 15, wherein the modification is a deletion on or before position 732 of SEQ ID NO: 1 of Solanum lycopersicum, or, in case of a crop other than tomato, on or around or before a position corresponding to position 732 of SEQ ID NO: 1 of Solanum lycopersicum, which modification leads to an premature stop codon in the FBXL13 protein.

17. Use of the modified FBXL13 gene of any of the paragraphs 1-8 for the development of a plant belonging to the Solanaceae family showing resistance to a virus belonging to the Torradovirus genus as compared to an isogenic plant belonging to the Solanaceae family not comprising the modified FBXL13 gene.

18. Use of the modified FBXL13 gene of any of the paragraphs 1-8 or a part thereof for identifying a plant belonging to the Solanaceae family showing resistance to a virus belonging to the Torradovirus genus as compared to an isogenic plant belonging to the Solanaceae family not comprising the modified FBXL13 gene.

19. Use of one or more of the sequences of SEQ ID NOS: 9 to 14 or a part thereof, or a marker derived thereof, as a marker for identifying a plant belonging to the Solanaceae family showing resistance to a virus belonging to the Torradovirus genus as compared to an isogenic plant belonging to the Solanaceae family not comprising the modified FBXL13 gene.

20. Use of any of the paragraphs 17 to 19, wherein the plant belongs to a species selected from the group consisting of Solanum melongena, Solanum lycopersicum, Solanum tuberosum and Capsicum annuum.

21. Method for producing a plant which exhibits resistance to a virus belonging to the Torradovirus genus, comprising reducing the endogenous level of FBXL13 protein in the plant by modifying an endogenous FBXL13 gene to become a non-functional FBXL13 gene.

22. Method of paragraph 21, wherein the modification is effected by mutagenic treatment of the seed, in particular by chemical or physical means.

23. Method of paragraph 21, wherein the modification is effected by CRISPR.

24. Method of paragraph 21, wherein reducing the endogenous level of FBXL13 protein in the plant is accomplished by reducing the expression of a FBXL13 gene of the plant by gene silencing or RNAi.

25. Method of any of the paragraphs 21 to 24, wherein the wild type FBXL13 gene has a nucleotide sequence according to SEQ ID NOS: 1-4.

26. A plant produced by a method described in any of the paragraphs 21 to 25.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A modified FBXL13 gene comprising a modification in the wild type FBXL13 nucleotide sequence of SEQ ID NOS:. 1-4 which leads to a change in the wild type FBXL13 amino acid sequence of SEQ ID NOS: 5-8, wherein said modified FBXL13 gene when present in the genome of a plant belonging to the Solanaceae family confers resistance to a virus belonging to the Torrado virus genus to said plant.

2. The modified FBXL13 gene of claim 1, wherein the modification of the nucleotide sequence is a SNP or indel that leads to a change in the amino acid sequence of the FBXL13 protein.

3. The modified FBXL13 gene of claim 1, wherein the modification of the nucleotide sequence of SEQ ID NOS: 1-4 leads to a premature STOP codon.

4. The modified FBXL13 gene of claim 1, wherein the modification of the nucleotide sequence of SEQ ID NOS: 1-4 leads to a substitution in the amino acid sequence of SEQ ID NOS:
5-8.

5. The modified FBXL13 gene of claim 1, wherein the modification of the nucleotide sequence is a deletion of the nucleotide on position 732 of the Solanum lycopersicum nucleotide sequence of SEQ ID NO: 1, or, for a crop other than Solanum lycopersicum, on a position corresponding to position 732 of the Solanum lycopersicum nucleotide sequence of SEQ ID NO:
1, leading to a truncated version of the Solanum lycopersicum amino acid sequence of SEQ ID NO: 5, or, for a crop other than Solanum lycopersicum, on a position corresponding to position 244 of the Solanum lycopersicum amino acid sequence of SEQ ID NO: 5.

6. The modified Solanum lycopersicum FBXL13 gene of claim 5, wherein the nucleotide sequence is depicted by SEQ ID NO: 9, which leads to the amino acid sequence of SEQ ID NO: 12.

7. The modified Solanum lycopersicum FBXL13 gene of claim 5, wherein the nucleotide sequence further comprises a SNP on position 731 of the Solanum lycopersicum nucleotide sequence of SEQ ID NO: 1, or, for a crop other than Solanum lycopersicum, on a position corresponding to position 731 of the Solanum lycopersicum nucleotide sequence of SEQ ID NO: 1, leading to an amino acid substitution on position 244 of the Solanum lycopersicum amino acid sequence of SEQ ID NO: 5, or, for a crop other than Solanum lycopersicum, on a position corresponding to position 244 of the Solanum lycopersicum amino acid sequence of SEQ ID NO: 5.

8. The modified Solanum lycopersicum FBXL13 gene of claim 7, wherein the nucleotide sequence is depicted by SEQ ID NO: 10, which leads to the amino acid sequence of SEQ ID NO: 13.

9. A plant belonging to the Solanaceae family, comprising the modified FBXL13 gene of claim 1.

10. The plant of claim 9, wherein the modified FBXL13 gene results in the plant showing resistance to a virus belonging to the Torradovirus genus as compared to an isogenic plant of the same species not comprising the modification of the FBXL13 gene.

11. The plant of claim 9, wherein the plant belongs to a species selected from the group consisting of Solanum melongena, Solanum lycopersicum, Solanum tuberosum and Capsicum annuum.

12. A seed of the plant of claim 9, comprising a modified FBXL13 gene.

13. A seed capable of developing into the plant of claim 9, comprising a modified FBXL13 gene.

14. A propagation material capable of developing into and/or being derived from the plant of claim 9, wherein the propagation material comprises the modified FBXL13 gene and wherein the propagation material is selected from a microspore, pollen, ovary, ovule, embryo, embryo sac, egg cell, cutting, root, hypocotyl, cotyledon, stem, leaf, flower, anther, seed, meristematic cell, protoplast, or cell, or tissue culture thereof.

15. A marker for identifying a plant belonging to the Solanaceae family showing resistance to a virus belonging to the Torradovirus genus, comprising the modified FBXL13 gene of claim 1 or a part thereof that comprises the modification.

16. The marker of claim 15, wherein the modification is a deletion on or before position 732 of SEQ ID NO: 1 of Solanum lycopersicum, or, in case of a crop other than tomato, on or around or before a position corresponding to position 732 of SEQ ID NO: 1 of Solanum lycopersicum, which modification leads to an premature stop codon in the FBXL13 protein.

17. A method for producing a plant which exhibits resistance to a virus belonging to the Torradovirus genus, comprising reducing the endogenous level of FBXL13 protein in the plant by modifying an endogenous FBXL13 gene to become a non-functional FBXL13 gene.

18. The method of claim 17, wherein the modification is effected by mutagenic treatment of the seed, in particular by chemical or physical means.

19. The method of claim 17, wherein the modification is effected by CRISPR.

20. The method of claim 17, wherein reducing the endogenous level of FBXL13 protein in the plant is accomplished by reducing the expression of a FBXL13 gene of the plant by gene silencing or RNAi.

21. The method of claim 17, wherein the wild type FBXL13 gene has a nucleotide sequence according to SEQ ID NOS: 1-4.

22. A plant produced by the method of claim 17.


SEQUENCE LISTING (14 sequences)

SEQ ID NO: 1 - DNA, 1296 bp, Solanum lycopersicum
  Solyc04g079810_WT_CDS
atgagtgtaatgaagcaacatataccatccaatttcctttgcaatgcagaacttgaggtggacgatatcagcaacttaccagcccagattgtcgacaagattctgtctcatttgtcacttagggatgctgtgaggacaagtgtcttgtcaagtaaatggagaaacaaatgggttactcttccaaaccttgtatttgacaatcaatctcttttgatctcatcccaagaccaaaccttcataaaaaataagatagtaaacattgttgatcatgttcttttacttcattctggtcccatacacaagttcaagctttctcatcgggatcttcaaggggtgtgtgatattgatagatggattctctttctatcaaggggtgctgtgaaggagtttattcttgaaatatggaaaggacatcgctacaaactccactcttctatatatctttgtcaaaagttgaaccacttggagctttttaattgtcttctaaaaccacctcacacatttaatggttttaaaagcttgaaaagcctcgatcttcagcacatcactatggaacaggatgcatttgagcaacttgtgtcgagatgccatttacttgagcggcttacactgatgaattttgagggcttctccgatcttaaaatccacgcaccgaatctcctcttctttgatgttggaggtgtctttgaagacatcaatttcatgaacacattcaatcttgctatcgtttatattgggctatatgtaaatcctggatttgacaaaaatcttactctaggcagtgctggaaatttggtcaagttttttgctcatttgcctcgtcttcaaaggcttgaagtacagagctttttcttgaagtatctggctgacaataaagtgccaggaagactacctacaccttgtgatgagctaagttttctttcaatgcgcataaatttcaaccatttggatgagtgtctggcagcactttgccttctcagaagttcccctaacctacaagagcttgagatgttggcacgcacagaagaacaaagtactttgagaaccgttgccagtgttatgaaagaggattaccagaattgtatgttcaatcaattgaggcatgttaagattgctggtataactgggcttaaacaagagctaaatttcgtcaattttctgctttcaaattcacctgttcttgaaagaatgacagtcaagccagcttcagttgacggtgcatgggagatgctaaaagagttgctacgcttcaggagagcttctgtacaagctgaaatcgtttacgttgacccataa

SEQ ID NO: 2 - DNA, 1188 bp, Solanum melongena
  Sme2.5_01177.1_WT_CDS
atggacactgattcggatagagatttaataagtgatttgcctcaaagtatcatagaaagcatcctcataagagtcccattactcgatgctgtaaggacaagcatattgtcaagaaaatggagatacaagtgggcaaccattacacaacttgtttttagtgacacatgtctgactccttgccatgacaaatcaattatcagttgcaatcttgtaaatttcattacccgttgcctgtttcttcatgatggaccaatccacaaatttgagttgaatacttcttactcgccaacttctcctgatttagatcaatggctacttttcctttctcgtaaagatatcaaagagttgattattgatataggagaagatgactggtttagagcaccttcgtgtttgttcttttgtcctaagttgactcatttggtgcttgttcgatgtgaattaaacccccctccaaatttcaaaggcttcttgtgtttgaagcaccttagtctccagcaagttatcattcctccacatgatattgaagtcctgatttccagttgccctcttcttgaaagcttgacattatcatattttgacagtttggagcttactattcaagctccaaatctcaaatacttgaatttggaagctgagcactttgaacaaagctcaggttgcaattttggcaagtttcttggtggtgttcctcgccttgagaggcttattggccatatttacttcactaaatatttgagtataggtaatgaggaaggaagctttcccgttatgtatcaaaatctgaagttcattgaactgtaccaagttagctttgaagacatgaaagagttactagttgtgcttcgcttgatcgtgagctcgcctaatctagaggagcttcaaatatcaagttcttcaattacaactaccactgatatatatgatctggaattttgggagaaagactggcctggtgactgcacttttggtaaactgaagattgtacaaatgactgatttctccggtctgcctcatgaaatcgcgtttatcaaattcttacttggacattcacctgttcttgaacaaatgattgttgctcctactatatatgtcacggataaagtggtgaaaatgttgatcgacttgttgacttttcgacgagcttctcctcaagctactgttaaatttgttcaagagccattgtag

SEQ ID NO: 3 - DNA, 1269 bp, Solanum tuberosum
  Sotub04g033070.1.1_WT_CDS
atggacactgattcggatagagatttattaagtgatttgcctcaaagtatcatagaaagcatcctcgtaaaagttccattagtcgatgctgtaaggacaagcatattgtcaagaaaatggagatacaagtgggcagccattacagaacttgtttttaatgacacatgtcggacttctggccatgacaaatcaattatcagttgcaatcttgtaaatttcattacccggtgcctgtttcttcatgacggaccgattcacaaatttgaattgaatacttcctactcaccagcttctcctgatttagatcaatggctacttttcctttctcgtaaagatatcaaagagttgattattgatataggagaagatgactggtttagagcaccttcatgtgtgttcttttgtcctaagttgactcatttggtgcttgttcgatgtgaattaaaccctcctccaaatttcaaaggcttcttgtgtttgaagcaccttagtctccagcaagttatcattcctccacatgatattgaagttctcatctccagttgtcctcttctcgagagcttgacattgtcatattttgacagtttggagcttactattcgagctccaaatctcaaatatctgaatttggaaggtgaatttaaggatatacgccttgagaatactccacatctgattggtatttcagttgccatgtatatgactgatgatatagctgagcactttgaacaatgctcaggttgcaatttcgacaagtttcttggtggtgttccttgccttgagaggcttattggtcatatatacttcactaaatatttgagtataggaaatgagcaagggaactttcccgttacgtatcaaaatctgaagttcattgaactgtaccaagttagttttgaagacatgaaagagttacttgttgtgcttcgcttgattgtgagttctcctaatctagaggagcttcaaatatctagttcctcgattacaaccaccaccgatatttatgatctagaattttgggagagagactggcctgctgactgcactttcggtaaactgaagattgtgcatatgactgatttctccggtctgccacatgaaatcgcgtttatcaaattcttacttggacattcacctgttctcgaacaaatgattgttgctcctactgtatatgtcacagataaagtggtgaaaatgttgatcgacctgttaacatttcgacgggcttctcctcaagctacagttaaatttattcaagagccattgtag

SEQ ID NO: 4 - DNA, 1314 bp, Capsicum annuum
  CA04g19850_WT_CDS
atgaatttcttcttctttgttactatgcagaagcaacgtattccatccaacttcccttgcaatgccgaacttgaggtggacaagatcagcaacttaccagcacagattgttgacaagattttgtctcatttgtcactcagggatgctgtgaggacaagcgtcttgtccagtaaatggagatacaaatgggtttctcttccaaaccttgtatttgacaatcaatctcttttgatttcatcccaagaccaaaccttcataaaaaataagatagttaacattgttgatcatgttctcttacttcattctggtcccatacaaaagttcaagctttctcatcgggatcttcaaggggtgtgtgatattgatagatggattctttttctatcaaggggttctgtgaaggaatttattcttgaaatatggaaaggacatcgctacaaactccattcttctatatatctttgtcaaaagttgatccacttggagctatttaattgtcttctaaaacctcctctcacattcattggttttaaaagtttgaaaagcctcgatcttcagcacatcactatggaacaagatgcatttgagcatctcgtatcgagctgccatttgcttgagcggcttacactgatgaattttgagggtttctccgatcttaaaatccatgcaccaaatctcctcttctttgacgttggaggcgtctttgaagacatcaatttcatggacacattcaaccttgccatagtttctattgggttatatgtgaatcctggatttgacaaaaatcttactctaggcagtgctggaaacttggtcaagttttttgctcatttacctcgtcttcaaaggctcgaagtacagagctttttcttgaagtatctggctgatggtaaggtaccgggaagactacctacgccttgtgatgagcttagttttctttcaatacgcataaatttcaaccatctggatgagtgtctggctgcactttgtcttctcagaagttcccctaacctacaggagcttgagatgttggcacgcacagaagaacaaagtgctttgagaaccgttgccagtgtcatggaagaaaactaccagaattgtatgtttaatcaattgcggcatgtgaagattgctggtatatctgggcttaaacaggagctaaatttcatcaattttctgcttgcaaattcacctattcttgaaaggatgacagttaagccagcttcagtagacggtgcatgggatatgctaaaagagttgcttcgctttaggagagcttcagtacaagccgagatcgtttaccttgacccttaa

SEQ ID NO: 5 - protein, 430 aa, Solanum lycopersicum
  Solyc04g079810_WT_protein
MSVMKQHIPSNFLCNAELEVDDISNLPAQIVDKILSHLSLRDAVRTSVLSSKWRNKWVTLPNLVFDNQSLLISSQDQTFIKNKIVNIVDHVLLLHSGPIHKFKLSHRDLQGVCDIDRWILFLSRGAVKEFILEIWKGHRYKLHSSIYLCQKLNHLELFNCLLKPPHTFNGFKSLKSLDLQHITMEQDAFEQLVSRCHLLERLTLMNFEGFSDLKIHAPNLLFFDVGGVFEDINFMNTFNLAIVYIGLYVNPGFDKNLTLGSAGNLVKFFAHLPRLQRLEVQSFFLKYLADNKVPGRLPTPCDELSFLSMRINFNHLDECLAALCLLRSSPNLQELEMLARTEEQSTLRTVASVMKEDYQNCMFNQLRHVKIAGITGLKQELNFVNFLLSNSPVLERMTVKPASVDGAWEMLELLRFRRASVQAEIVYVDP

SEQ ID NO: 6 - protein, 395 aa, Solanum melongena
  Sme2.5_01177.1_WT_protein
MDTDSDRDLISDLPQSIIESILIRVPLLDAVRTSILSRKWRYKWATITQLVFSDTCLTPCHDKSIISCNLVNFITRCLFLHDGPIHKFELNTSYSPTSPDLDQWLLFLSRKDIKELIIDIGEDDWFRAPSCLFFCPKLTHLVLVRCELNPPPNFKGFLCLKHLSLQQVIIPPHDIEVLISSCPLLESLTLSYFDSLELTIQAPNLKYLNLEAEHFEQSSGCNFGKFLGGVPRLERLIGHIYFTKYLSIGNEEGSFPVMYQNLKFIELYQVSFEDMKELLVVLRLIVSSPNLEELQISSSSITTTTDIYDLEFWEKDWPGDCTFGKLKIVQMTDFSGLPHEIAFIKFLLGHSPVLEQMIVAPTIYVTDKVVKMLIDLLTFRRASPQATVKFVQEPL

SEQ ID NO: 7 - protein, 422 aa, Solanum tuberosum
  Sotub04g033070.1.1_WT_protein
MDTDSDRDLLSDLPQSIIESILVKVPLVDAVRTSILSRKWRYKWAAITELVFNDTCRTSGHDKSIISCNLVNFITRCLFLHDGPIHKFELNTSYSPASPDLDQWLLFLSRKDIKELIIDIGEDDWFRAPSCVFFCPKLTHLVLVRCELNPPPNFKGFLCLKHLSLQQVIIPPHDIEVLISSCPLLESLTLSYFDSLELTIRAPNLKYLNLEGEFKDIRLENTPHLIGISVAMYMTDDIAEHFEQCSGCNFDKFLGGVPCLERLIGHIYFTKYLSIGNEQGNFPVTYQNLKFIELYQVSFEDMKELLVVLRLIVSSPNLEELQISSSSITTTTDIYDLEFWERDWPADCTFGKLKIVHMTDFSGLPHEIAFIKFLLGHSPVLEQMIVAPTVYVTDKVVKMLIDLLTFRRASPQATVKFIQEPL

SEQ ID NO: 8 - protein, 437 aa, Capsicum annuum
  CA04g19850_WT_protein
MNFFFFVTMQKQRIPSNFPCNAELEVDKISNLPAQIVDKILSHLSLRDAVRTSVLSSKWRYKWVSLPNLVFDNQSLLISSQDQTFIKNKIVNIVDHVLLLHSGPIQKFKLSHRDLQGVCDIDRWILFLSRGSVKEFILEIWKGHRYKLHSSIYLCQKLIHLELFNCLLKPPLTFIGFKSLKSLDLQHITMEQDAFEHLVSSCHLLERLTLMNFEGFSDLKIHAPNLLFFDVGGVFEDINFMDTFNLAIVSIGLYVNPGFDKNLTLGSAGNLVKFFAHLPRLQRLEVQSFFLKYLADGKVPGRLPTPCDELSFLSIRINFNHLDECLAALCLLRSSPNLQELEMLARTEEQSALRTVASVMEENYQNCMFNQLRHVKIAGISGLKQELNFINFLLANSPILERMTVKPASVDGAWDMLKELLRFRRASVQAEIVYLDP

SEQ ID NO: 9 - DNA, 732 bp, Solanum lycopersicum
  Solyc04g079810_deletion_CDS
atgagtgtaatgaagcaacatataccatccaatttcctttgcaatgcagaacttgaggtggacgatatcagcaacttaccagcccagattgtcgacaagattctgtctcatttgtcacttagggatgctgtgaggacaagtgtcttgtcaagtaaatggagaaacaaatgggttactcttccaaaccttgtatttgacaatcaatctcttttgatctcatcccaagaccaaaccttcataaaaaataagatagtaaacattgttgatcatgttcttttacttcattctggtcccatacacaagttcaagctttctcatcgggatcttcaaggggtgtgtgatattgatagatggattctctttctatcaaggggtgctgtgaaggagtttattcttgaaatatggaaaggacatcgctacaaactccactcttctatatatctttgtcaaaagttgaaccacttggagctttttaattgtcttctaaaaccacctcacacatttaatggttttaaaagcttgaaaagcctcgatcttcagcacatcactatggaacaggatgcatttgagcaacttgtgtcgagatgccatttacttgagcggcttacactgatgaattttgagggcttctccgatcttaaaatccacgcaccgaatctcctcttctttgatgttggaggtgtctttgaagacatcaatttcatgaacacattcaatcttgctatcgtttaa

SEQ ID NO: 10 - DNA, 747 bp, Solanum lycopersicum
  Solyc04g079810_deletion+SNP_CDS
atgagtgtaatgaagcaacatataccatccaatttcctttgcaatgcagaacttgaggtggacgatatcagcaacttaccagcccagattgtcgacaagattctgtctcatttgtcacttagggatgctgtgaggacaagtgtcttgtcaagtaaatggagaaacaaatgggttactcttccaaaccttgtatttgacaatcaatctcttttgatctcatcccaagaccaaaccttcataaaaaataagatagtaaacattgttgatcatgttcttttacttcattctggtcccatacacaagttcaagctttctcatcgggatcttcaaggggtgtgtgatattgatagatggattctctttctatcaaggggtgctgtgaaggagtttattcttgaaatatggaaaggacatcgctacaaactccactcttctatatatctttgtcaaaagttgaaccacttggagctttttaattgtcttctaaaaccacctcacacatttaatggttttaaaagcttgaaaagcctcgatcttcagcacatcactatggaacaggatgcatttgagcaacttgtgtcgagatgccatttacttgagcggcttacactgatgaattttgagggcttctccgatcttaaaatccacgcaccgaatctcctcttctttgatgttggaggtgtctttgaagacatcaatttcatgaacacattcaatcttgctatcgtttcattgggctatatgtaa

SEQ ID NO: 11 - DNA, 1296 bp, Solanum lycopersicum
  Solyc04g079810_SNP_CDS
atgagtgtaatgaagcaacatataccatccaatttcctttgcaatgcagaacttgaggtggacgatatcagcaacttaccagcccagattgtcgacaagattctgtctcatttgtcacttagggatgctgtgaggacaagtgtcttgtcaagtaaatggagaaacaaatgggttactcttccaaaccttgtatttgacaatcaatctcttttgatctcatcccaagaccaaaccttcataaaaaataagatagtaaacattgttgatcatgttcttttacttcattctggtcccatacacaagttcaagctttctcatcgggatcttcaaggggtgtgtgatattgatagatggattctctttctatcaaggggtgctgtgaaggagtttattcttgaaatatggaaaggacatcgctacaaactccactcttctatatatctttgtcaaaagttgaaccacttggagctttttaattgtcttctaaaaccacctcacacatttaatggttttaaaagcttgaaaagcctcgatcttcagcacatcactatggaacaggatgcatttgagcaacttgtgtcgagatgccatttacttgagcggcttacactgatgaattttgagggcttctccgatcttaaaatccacgcaccgaatctcctcttctttgatgttggaggtgtctttgaagacatcaatttcatgaacacattcaatcttgctatcgtttctattgggctatatgtaaatcctggatttgacaaaaatcttactctaggcagtgctggaaatttggtcaagttttttgctcatttgcctcgtcttcaaaggcttgaagtacagagctttttcttgaagtatctggctgacaataaagtgccaggaagactacctacaccttgtgatgagctaagttttctttcaatgcgcataaatttcaaccatttggatgagtgtctggcagcactttgccttctcagaagttcccctaacctacaagagcttgagatgttggcacgcacagaagaacaaagtactttgagaaccgttgccagtgttatgaaagaggattaccagaattgtatgttcaatcaattgaggcatgttaagattgctggtataactgggcttaaacaagagctaaatttcgtcaattttctgctttcaaattcacctgttcttgaaagaatgacagtcaagccagcttcagttgacggtgcatgggagatgctaaaagagttgctacgcttcaggagagcttctgtacaagctgaaatcgtttacgttgacccataa

SEQ ID NO: 12 - protein, 243 aa, Solanum lycopersicum
  Solyc04g079810_deletion_protein
MSVMKQHIPSNFLCNAELEVDDISNLPAQIVDKILSHLSLRDAVRTSVLSSKWRNKWVTLPNLVFDNQSLLISSQDQTFIKNKIVNIVDHVLLLHSGPIHKFKLSHRDLQGVCDIDRWILFLSRGAVKEFILEIWKGHRYKLHSSIYLCQKLNHLELFNCLLKPPHTFNGFKSLKSLDLQHITMEQDAFEQLVSRCHLLERLTLMNFEGFSDLKIHAPNLLFFDVGGVFEDINFMNTFNLAIV

SEQ ID NO: 13 - protein, 248 aa, Solanum lycopersicum
  Solyc04g079810_ deletion+SNP_protein
MSVMKQHIPSNFLCNAELEVDDISNLPAQIVDKILSHLSLRDAVRTSVLSSKWRNKWVTLPNLVFDNQSLLISSQDQTFIKNKIVNIVDHVLLLHSGPIHKFKLSHRDLQGVCDIDRWILFLSRGAVKEFILEIWKGHRYKLHSSIYLCQKLNHLELFNCLLKPPHTFNGFKSLKSLDLQHITMEQDAFEQLVSRCHLLERLTLMNFEGFSDLKIHAPNLLFFDVGGVFEDINFMNTFNLAIVSLGYM

SEQ ID NO: 14 - protein, 431 aa, Solanum lycopersicum
  Solyc04g079810_SNP_protein
MSVMKQHIPSNFLCNAELEVDDISNLPAQIVDKILSHLSLRDAVRTSVLSSKWRNKWVTLPNLVFDNQSLLISSQDQTFIKNKIVNIVDHVLLLHSGPIHKFKLSHRDLQGVCDIDRWILFLSRGAVKEFILEIWKGHRYKLHSSIYLCQKLNHLELFNCLLKPPHTFNGFKSLKSLDLQHITMEQDAFEQLVSRCHLLERLTLMNFEGFSDLKIHAPNLLFFDVGGVFEDINFMNTFNLAIVSIGLYVNPGFDKNLTLGSAGNLVKFFAHLPRLQRLEVQSFFLKYLADNKVPGRLPTPCDELSFLSMRINFNHLDECLAALCLLRSSPNLQELEMLARTEEQSTLRTVASVMKEDYQNCMFNQLRHVKIAGITGLKQELNFVNFLLSNSPVLERMTVKPASVDGAWEMLKELLRFRRASVQAEIVYVDP